(19) United States
(12) Patent Application Publication
(10) Pub. No.: US 2022/0054309 A1
(43) Pub. Date: Feb. 24, 2022

(54) GLAUCOMA TREATMENT METHODS

(71) Applicants: JAIE CHIA-HSIN KUO, LAKE FOREST, CA (US); RUTH ILING KUO, LAKE FOREST, CA (US)

(72) Inventors: JAIE CHIA-HSIN KUO, LAKE FOREST, CA (US); RUTH ILING KUO, LAKE FOREST, CA (US)

(21) Appl. No.: 17/000,288

(22) Filed: Aug. 22, 2020

(51) Int. Cl. A61F 9/00; A61F 9/007; A61B 18/02; A61B 18/04

(52) U.S. Cl. CPC A61F 9/0017; A61F 9/00736; A61F 2250/0013; A61B 18/04; A61F 2250/0003; A61B 18/02

(57) ABSTRACT

A method of surgically altering trabecular meshwork of an eye to create a throughput hole on the meshwork without an implant or a permanent stent comprising inserting an applicator into the meshwork, wherein the applicator has a meshwork-contacting element at its tip section to contact the meshwork and provide energy or mechanical force to the contacted trabecular meshwork.

### CROSS-REFERENCE TO RELATED APPLICATION

This application claims the benefits of a Provisional patent application Ser. No. 62/923,480, filed Oct. 19, 2019, the entire contents of which are incorporated herein by reference.

### BACKGROUND OF THE INVENTION

### Field of the Invention

This invention relates to methods of reducing elevated pressure in organs of the human body. More precisely, the invention relates to the treatment of glaucoma by trabecular bypass surgery and associated devices, which utilizes mechanical forces, heat energy and/or cryogenic energy to ablate the trabecular meshwork and secure at least one hole into the diseased trabecular meshwork in order to restore the existing aqueous outflow pathways of the eye.

### Background of the Invention

Glaucoma affects 60 million people worldwide and about 2% in America alone. Glaucoma is a form of eye disease that causes loss of vision if left untreated. Intraocular pressure elevation is the main factor in all glaucomas.

The main source of resistance to outflow in glaucoma associated with eye pressure elevation is located in the trabecular meshwork. The tissue located in the meshworks allows the “aqueous” to enter into Schlemm's canal, which is then transferred into aqueous collector channels in the posterior wall of Schlemm's canal and then into aqueous veins. The aqueous is a transparent liquid that fills the region between the cornea at the front of the eye and the lens. It is constantly secreted by the ciliary body around the lens in a continuous flow to the eye's front chamber.

The pressure in the eye is determined by a balance between the production of the aqueous and its exit through the trabecular meshwork or uveal scleral outflow. The meshwork is located in the outer rim of the iris and the internal periphery of the cornea. The meshwork adjacent to Schlemm's canal causes most of the resistance to aqueous outflow.

Glaucoma is generally classified into two classes: open-angle glaucoma and closed-angle glaucoma. Open-angle refers to any glaucoma in which the angle of the anterior chamber remains open, but the aqueous exit through the trabecular meshwork is blocked. The cause of the block in the trabecular meshwork is unknown for most cases; however, there are secondary open-angle glaucomas that can cause swelling of the trabecular spaces, abnormalities in pigment dispersion, or diseases that produce vascular congestion.

Closed-angle glaucoma is caused by the closure of the anterior angle by contact between the iris and the inner surface of the trabecular meshwork. This prevents the normal drainage of the aqueous from the anterior chamber in the eye.

Current therapies for glaucoma directly decrease the intraocular pressure. Initially, it was treated with medical therapy with drops or pills that decrease production of the aqueous or increase outflow. Many of these drug therapies are partnered with side effects, such as headache, blurred vision, allergic reactions, death from cardiopulmonary complications and potential interaction with other drugs. If drug therapy fails, surgical therapy is utilized.

Surgical therapy for open-angle glaucoma utilizes laser (trabeculoplasty), trabeculectomy and aqueous shunting implants after failure of trabeculectomy or if trabeculectomy is unlikely to succeed. Trabeculectomy is a major surgery that is widely used and is augmented with topically applied anticancer drugs to decrease scarring and increase surgical success.

Around 100,000 trabeculectomies are performed on Medicare age patients per year in the United States. The number would increase if the morbidity associated could decrease. Current morbidity associated includes failures (10-15%), infection (2-5%), choroidal hemorrhage (1%, a severe internal hemorrhage from pressure too low resulting in visual loss), cataract formation, and hypotony maculopathy (potential reversible visual loss from pressure too low).

If it were possible to bypass the local resistance to outflow of aqueous at the point of the resistance and use existing outflow mechanisms, surgical morbidity would greatly decrease. The reason for this is that the episcleral aqueous veins have a back pressure that would prevent the eye pressure from going too low. This would substantially eliminate the risk of hypotony maculopathy and choroidal hemorrhage. Furthermore, visual recovery would be very rapid and risk of infection would be very small (from 2-5% to about 0.05%). These reasons are why surgeons have tried for decades to develop a workable surgery for the trabecular meshwork.

Previous techniques, such as goniotomy and trabeculotomy, have already been tried, as have other mechanical disruptions, like trabeculopuncture, goniophotoablation, laser trabecular ablation and goniocurretage. All of which have been seen to fail long term, or not demonstrated by trials to succeed.

Trabeculectomy is the most commonly performed filtering surgery, with Viscocanalostomy (VC) and non-penetrating trabeculectomy (NPT) being two new variations of such surgery. These are ab-externo (from the outside) major ocular procedures in which Schlemm's canal is surgically exposed by making a large and very deep scleral flap. In the VC procedure, Schlemm's canal is cannulated and a viscoelastic drug injected (which dilates Schlemm's canal and the aqueous collector channels). In the NPT procedure, the inner wall of Schlemm's canal is stripped off after surgically exposing the canal.

Trabeculectomy, VC, and NPT are performed under a conjunctival and scleral flap, such that the aqueous humor is drained onto the surface of the eye or into the tissues located within the lateral wall of the eye. Normal physiological outflows are not used. These surgical operations are major procedures with significant ocular morbidity. When Trabeculectomy, VC, and NPT are thought to have a low chance for success, a number of implantable drainage devices have been used to ensure that the desired filtration and outflow of the aqueous humor through the surgical opening will continue. The risk of placing a glaucoma drainage implant also includes hemorrhage, infection and postoperative double vision that is a complication unique to drainage implants.

Recently, there are ab-interno ocular procedures using a stent implant through the trabecular meshworks so to prevent the tissue of punctured trabecular meshworks from re-joining via tissue scaring or healing processes, whereas a healing process is known as filling in, which has a tendency to close surgically created openings in the trabecular meshwork. U.S. Pat. Nos. 6,638,239, 10,285,856, and others described the ab interno implants techniques for this procedure, which patents are referred herein by reference. There is a clinical need to maintain the punctured tissue of trabecular meshworks open without the assist of a stent or an implant for aqueous to continue outflow from the anterior chamber to Schlemm's canal and subsequently to the aqueous collector channel.

Radiofrequency ablation (RF) has been quite common in electrophysiology to provide energy to the electrodes as energy source. U.S. Pat. No. 6,104,952 teaches a device system for applying radiofrequency energy to treat a tissue, herein incorporated for reference. The device system comprising: (a) an elongated tubing having a distal section, a distal end, a proximal end, openings at both ends, and at least one lumen extending therebetween, the electrode means disposed at the distal end, wherein the electrode means has at least one surface zone for contacting the tissue, and (b) means for applying radiofrequency energy to the electrode means of the device, wherein radiofrequency energy is provided by a RF generator.

Of particular interest to the present invention are RF therapeutic protocols which have been proven to be highly effective as used by electrophysiologists for the treatment of tachycardia; by neurosurgeons for the treatment of Parkinson's disease; and by neurosurgeons and anesthetists for other RF procedures such as Gasserian ganglionectomy for trigeminal neuralgia and percutaneous cervical cordotomy for intractable pains. Other literature or patents for RF energy are cited herein for reference: Imran in U.S. Pat. No. 5,281,218 entitled “Catheter having needle electrode for radiofrequency ablation”, Edwards et al. in U.S. Pat. No. 5,456,662 entitled “Method for reducing snoring by RF ablation of the uvula”, and Wang et al. in U.S. Pat No. 10,265,122 entitled “Nerve ablation devices and related methods of use”.

### SUMMARY OF THE INVENTION

The trabecular meshwork and juxtacanalicular tissue together provide the majority of the resistance to the outflow of aqueous and, as such, are logical targets for surgical removal in the treatment of open-angle glaucoma. In addition, minimal amounts of tissue are altered while exiting physiologic outflow pathways are utilized. Trabecular bypass surgery has the potential for much lower risks of choroidal hemorrhage, infection while uses existing physiologic outflow mechanisms. This surgery could be performed under topical anesthesia in a physician's office with rapid visual recovery.

In summary, the invention relates to the treatment of glaucoma by trabecular bypass surgery and a transient device, which utilizes mechanical rotational forces, heat, and/or cryogenic energy to ablate the trabecular meshwork and secure (or result in) at least one hole or opening into the diseased trabecular meshwork in order to restore the existing outflow pathways. In one embodiment, the transient device is removed from the patient after the procedure is completed. In a further embodiment, the at least one hole or opening maintains its throughput space transiently, semi-permanently or permanently.

In one embodiment, the invention relates to a method of securing at least one aqueous hole or opening through trabecular meshwork of an eye, wherein the at least one aqueous hole is generated via a meshwork-contacting element in contact with the trabecular meshwork that ablates a portion of the trabecular meshwork. The generated aqueous hole of the present invention is an open throughput on trabecular meshwork that is large enough to allow aqueous to freely flow from the anterior chamber to Schlemm's canal lasting for clinically effective period. In other words, the pressure for aqueous to flow through the open throughput is substantially lower than the pressure of aqueous through the unaltered meshwork. In one embodiment, the meshwork-contacting element is a metallic member or electrode-like material. In a further embodiment, there is provided a RF current generator, wherein an electrical conductor is coupled from the RF current generator to the meshwork-contacting element for delivering RF current to the meshwork-contacting element for ablating the tissue of trabecular meshwork.

In another embodiment, the invention relates to a method of securing at least one aqueous opening through trabecular meshwork of an eye, wherein the at least one aqueous opening is generated via a meshwork-contacting element (for example, a rotational means, heating means, cryogenic means, or others) in contact with the trabecular meshwork that ablates a portion of the trabecular meshwork. The generated aqueous opening of the present invention via heat treatment, cryogenic treatment, rotational mechanic treatment, or combination thereof is at least one hole or open throughput on trabecular meshwork that has enough spacious room to allow aqueous to freely flow from the anterior chamber to Schlemm's canal for a therapeutic period according to a patient's need. The at least one hole on trabecular meshwork is to remain much longer than the holes created via, for example, traditional laser trabecular ablation having a non trabecular meshwork-contacting laser source.

In one embodiment, it is provided a method of surgically altering trabecular meshwork of an eye to create a throughput hole on the meshwork comprising: inserting a meshwork-contacting element of an applicator into the meshwork and contacting the meshwork, wherein the meshwork-contacting element provides energy or mechanical force to the contacted trabecular meshwork, and thereafter removing the applicator from the eye. The meshwork-contacting element of the present invention has different characteristics from that of a non meshwork-contacting element, such as a laser source.

In one embodiment, the meshwork-contacting element is located at a tip section of the applicator. In another embodiment, the applicator comprises a piercing member. In another embodiment, the piercing member is a self-trephining member.

In a further embodiment, the energy referred in this invention is heat energy, cryogenic energy, or alternating heat/cryogenic energy cycles.

In one embodiment, an exterior surface of the meshwork-contacting element is made of metallic material. In another embodiment, an exterior surface of the meshwork-contacting element is coated with a pharmaceutically active ingredient. In a further embodiment, an exterior surface of the meshwork-contacting element is porous or having perforated openings.

In one embodiment, an exterior surface of the meshwork-contacting element is a coarse surface so that when the meshwork-contacting element is rotated, the meshwork is partially ablated or cut away to create an opening or a throughput hole.

In one embodiment, it is provided a method of surgically delivering a pharmaceutical ingredient to trabecular meshwork of an eye comprising: inserting a meshwork-contacting element of an applicator into the meshwork and contacting the meshwork, wherein the surface of the meshwork-contacting element is coated with a pharmaceutical ingredient (or therapeutic drug) that can be released once contacting the target trabecular meshwork site. In another embodiment, the drug can be releasably stored within the meshwork-contacting element. Once at the target trabecular meshwork site, the therapeutic drug is released. In this particular embodiment, the meshwork-contacting element may be porous or equipped with a plurality of small holes or openings for releasing the drug as desired. Thereafter, the applicator is removed from the eye.

In one embodiment, it is provided a method of surgically delivering a pharmaceutical ingredient to trabecular meshwork of an eye comprising: inserting a meshwork-contacting element of a hollow applicator into the meshwork and contacting the meshwork, wherein a surface-medicated balloon is inserted through a lumen of the applicator to the meshwork-contacting element, wherein the surface-coated pharmaceutical ingredient (or therapeutic drug) is released by inflating the balloon outwardly toward the contacted meshwork, and thereafter removing the applicator from the eye. In this particular embodiment, the meshwork-contacting element may be equipped with a plurality of small holes or openings for allowing the balloon surface to contact the target meshwork.

In one embodiment, a method of surgically altering trabecular meshwork of an eye to create at least one throughput hole on said meshwork comprising: inserting a meshwork-contacting element of an applicator into said meshwork and contacting said meshwork, wherein said meshwork-contacting element provides energy or mechanical force to the contacted trabecular meshwork, and thereafter removing the applicator from the eye.

In one embodiment, it is provided wherein said meshwork-contacting element is located at a tip section of said applicator.

In one embodiment, it is provided wherein said energy is heat energy, cryogenic energy, or alternating heat and cryogenic cycles.

In one embodiment, it is provided wherein the applicator further comprises a piercing member.

In one embodiment, it is provided wherein the piercing member is a self-trephining member.

In one embodiment, it is provided wherein an exterior surface of the meshwork-contacting element is made of metallic material or energy generating material.

In one embodiment, it is provided wherein an exterior surface of the meshwork-contacting element is coated with a pharmaceutical ingredient.

In one embodiment, it is provided wherein an exterior surface of the meshwork-contacting element is porous or having perforated openings.

In one embodiment, it is provided wherein an exterior surface of the meshwork-contacting element is a coarse surface or having sharp points so that when the meshwork-contacting element is rotated, the meshwork is ablated or cut away to create the throughput hole.

Alternatively, a method of surgically altering trabecular meshwork of an eye to create at least one throughput hole on said meshwork comprising: inserting a meshwork-contacting element of an applicator into said meshwork and contacting said meshwork, wherein a surface-medicated balloon is inserted through a lumen of the applicator to the meshwork-contacting element region, wherein the surface-coated pharmaceutical ingredient (or therapeutic drug) is released by inflating the balloon outwardly toward the contacted meshwork, and thereafter removing the applicator from the eye.

### BRIEF DESCRIPTION OF THE DRAWINGS

Following the explanation of the inventions, certain embodiments and modifications thereof will be shown within the detailed descriptions in reference to the figures that follow:

FIG. 1 is a sectional view of an eye for illustration purposes.

FIG. 2 is a close-up sectional view showing the anatomical diagram of trabecular meshwork and the anterior chamber of the eye from FIG. 1.

FIG. 3 is an angled view of the solid meshwork-contacting element used to keep the punctured meshwork open having features of the inner workings.

FIG. 3A is an angled view of the mesh-like cover meshwork-contacting element used to keep the punctured meshwork open.

FIG. 3B is an angled view of the striped open and closed cover meshwork-contacting element used to keep the punctured meshwork open.

FIG. 3C is an angled view of the rough surface meshwork-contacting element used to keep the punctured meshwork open.

FIG. 4 is a top view of the meshwork-contacting element from FIG. 3.

FIG. 5 is a schematic drawing of a flat side view of the element from FIG. 3, once inserted into and through the trabecular meshwork.

FIG. 6 shows the applicator with the meshwork-contacting element at its tip ready to be inserted into and through the trabecular meshwork of an eye ab-interno.

FIG. 7 is an applicator with a meshwork-contacting element of the present invention.

FIG. 8 is a cross-sectional view of the applicator of FIG. 7.

FIG. 9 is the applicator with the power button, light indicator and/or the rotational button.

FIG. 10 is a view of the applicator including inner workings at the base of the applicator.

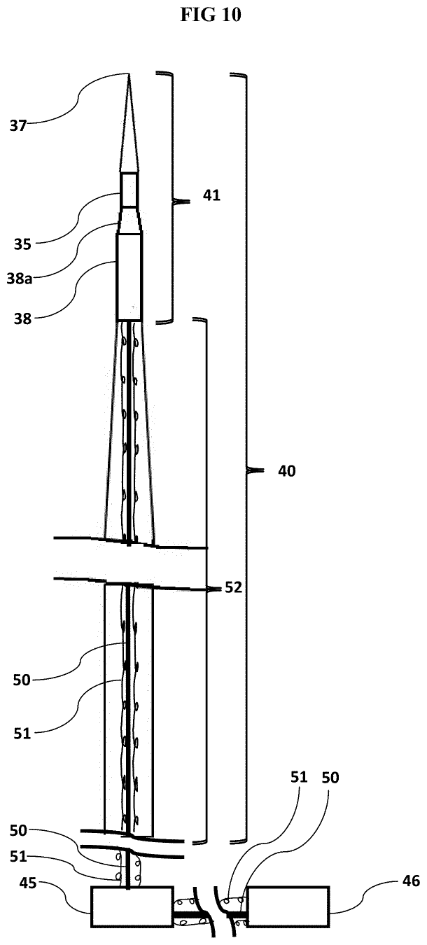

### DETAILED DESCRIPTION OF THE PREFERRED EMBODIMENTS

The drawings overall demonstrate a method for treating glaucoma, both open-angled and closed angle, via trabecular bypass surgery. In particular, the meshwork-contacting element of the device is transiently used to bypass the diseased or deficient trabecular meshwork in order to restore or create outflow pathways; and methods therefore are disclosed. After the procedure, no device or stent is left in the eye. The device is also generally known as the applicator or handpiece in the invention.

While the following descriptions set forth the device's specifics, it is not to be interpreted as the invention's limitations. Variations of the applications and modifications are also encompassed in the general concepts.

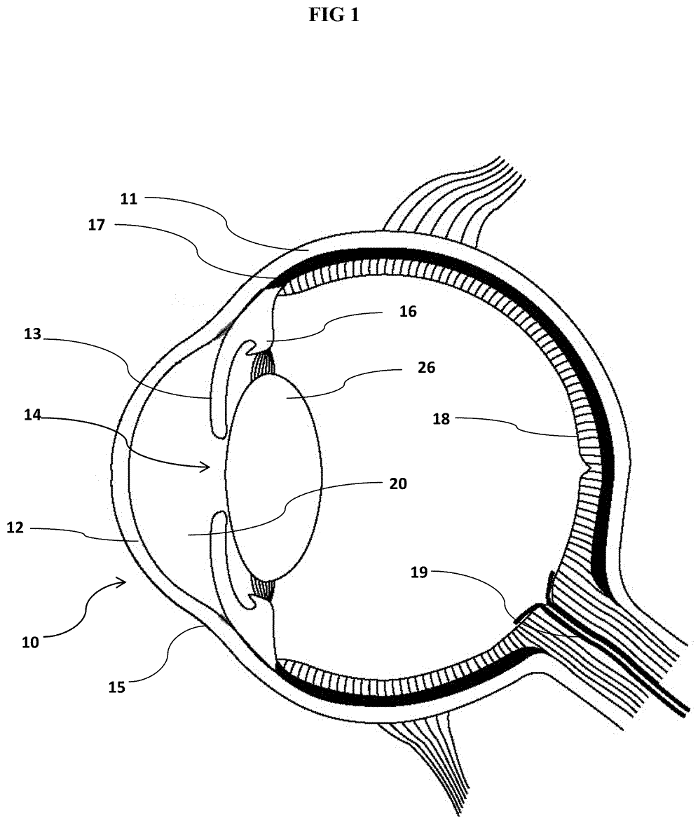

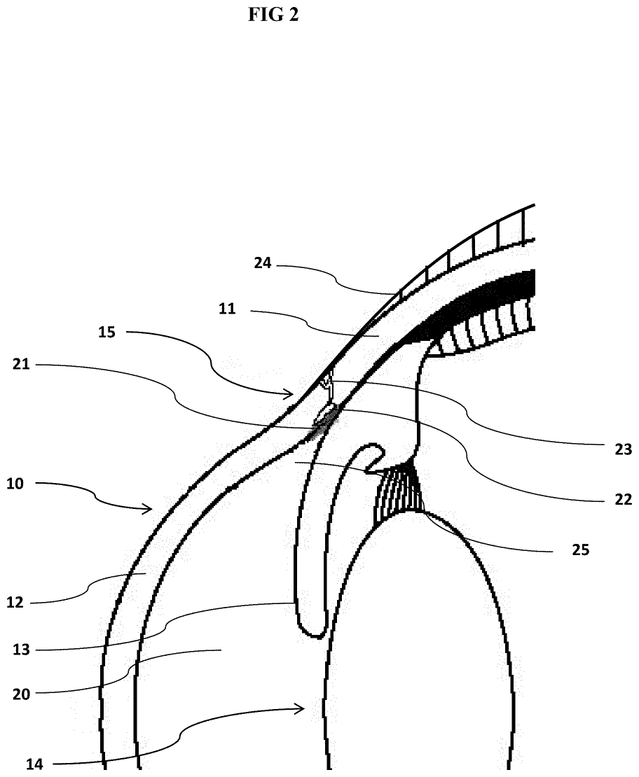

For background information and illustration purposes, FIG. 1 shows a sectional view of an eye 10, while FIG. 2 shows a close-up view, showing the relative anatomical locations of a trabecular meshwork 21, an anterior chamber 20, and Schlemm's canal 22. Thick collagenous tissue known as sclera 11 covers the entire eye 10 except that portion covered by the cornea 12. The cornea 12 is a thin transparent tissue that focuses and transmits light into the eye and through a pupil 14 which is generally a circular hole in the center of an iris 13 (colored portion of the eye). The cornea 12 merges into the sclera 11 at a juncture referred to as a limbus 15. A ciliary body 16 begins internally in the eye and extends along the interior of the sclera 11 and is coextensive with a choroid 17. The choroid 17 is a vascular layer of the eye, located between the sclera 11 and an underlying retina 18. An optic nerve 19 transmits visual information to the brain and is the anatomic structure that is progressively destroyed by glaucoma.

The anterior chamber 20 of the eye 10 (FIGS. 1 and 2), which is bounded anteriorly by the cornea 12 and posteriorly by the iris 13 and a lens 26, is filled with aqueous humor (also herein referred to as “aqueous”). Aqueous is produced primarily by the ciliary body 16 and reaches an anterior chamber angle 25, formed between the iris 13 and the cornea 12, through the pupil 14.

Referring in particular to FIGS. 1 and 2, in a normal eye, aqueous is removed from the anterior chamber 20 through the trabecular meshwork 21. Aqueous passes through trabecular meshwork 21 into Schlemm's canal 22 and thereafter through a plurality of aqueous veins 23, which merge with blood-carrying veins, and into systemic venous circulation. Intraocular pressure (IOP) of the eye 10 is maintained by an intricate balance between secretion and outflow of aqueous in the manner described above. Glaucoma is, in most cases, characterized by an excessive buildup of aqueous fluid in the anterior chamber 20 which leads to an increase in intraocular pressure. Fluids are relatively incompressible, and thus intraocular pressure is distributed relatively uniformly throughout the eye 10.

As shown in FIG. 2, the trabecular meshwork 21 is adjacent a small portion of the sclera 11. Exterior to the sclera 11 is a conjunctiva 24. Traditional procedures that create a hole or opening for implanting a device through the tissues of the conjunctiva 24 and sclera 11 involve extensive surgery.

The trabecular meshwork is an area of tissue in the eye located around the base of the cornea, near the ciliary body, and is responsible for draining the aqueous humor from the eye via the anterior chamber (the chamber on the front of the eye covered by the cornea). The tissue is spongy and lined by trabeculocytes; it allows fluid to drain into a set of tubes called Schlemm's canal flowing into the blood system. Since trabecular meshwork is spongy which is fundamentally different from other tissues of the human body, it would not be obvious to one ordinary skilled in the art to think of using or applying an energy source or mechanical force to ablate trabecular meshwork to generate a spacious throughput (or holes) on the meshwork to enhance aqueous outflow from the anterior chamber.

The meshwork is generally divided up into three parts, with characteristically different ultrastructures: inner uveal meshwork, corneoscleral meshwork, and juxtacanalicular tissue (also known as the cribriform meshwork). The trabecular meshwork is assisted to a small degree in the drainage of aqueous humor by a second outflow pathway, the uveo-scleral pathway (5-10% of outflow occurs this way). The uveo-scleral pathway is increased with the use of glaucoma drugs such as prostaglandins (e.g., Xalatan, Travatan).

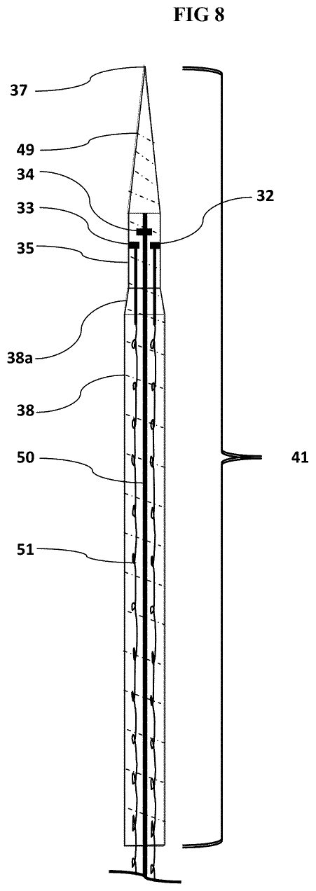

Surgical methods and relates medical devices for treating glaucoma are disclosed. The method of the present invention comprises trabecular bypass surgery, which involves bypassing diseased trabecular meshwork with the use of a sharp piercing member 49 of an applicator 41, followed by a heat/cryogenic method and/or mechanical rotation force. The device/applicator is inserted into the trabecular meshwork by a piercing member that is slidably advanced through the meshwork, optionally with slight rotation of the piercing member. The rotational mechanism can be found in U.S. Pat. No. 6,102,908, herein cited for reference. In one embodiment as shown in FIG. 8, the applicator 41 further comprises a rotating member 50 extending through the tubular shaft 51 and being rotatable within the tubular shaft, wherein a meshwork-contacting element 35 is optionally connected to the rotating member 50.

The meshwork-contacting element 35 is herein defined in this invention as the element on an applicator 41 to initiate or assist creating a hole (or a spacious throughput) on the trabecular meshwork 21. In a preferred embodiment, the rotating member is manually turned in clockwise or counter-clockwise manner to exert a rotational movement of the meshwork-contacting element at the tip section. In one embodiment, certain non-manual mechanism can be established with the rotating member. The applicator is exposed to the anterior chamber of the eye, allowing fluid collection channels at about an exterior surface of the trabecular meshwork or up to the level of aqueous veins, whereas the meshwork-contacting element of the applicator is positioned through the trabecular meshwork intimately. Some embodiments relate to a method of increasing aqueous humor outflow in an eye of a patient to reduce the intraocular pressure (IOP) therein. In one embodiment, the method comprises bypassing diseased or deficient trabecular meshwork at the level of the trabecular meshwork and thereby complementing existing outflow pathways.

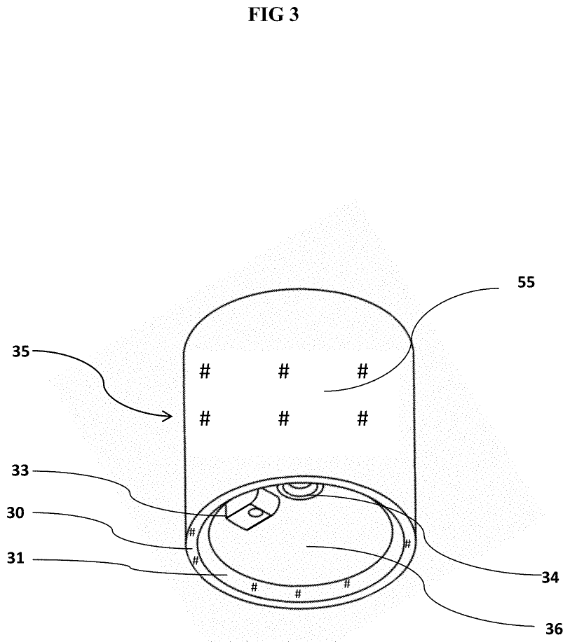

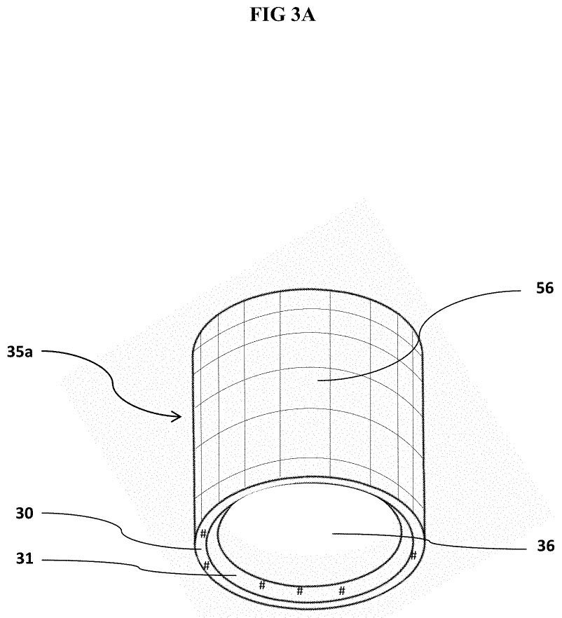

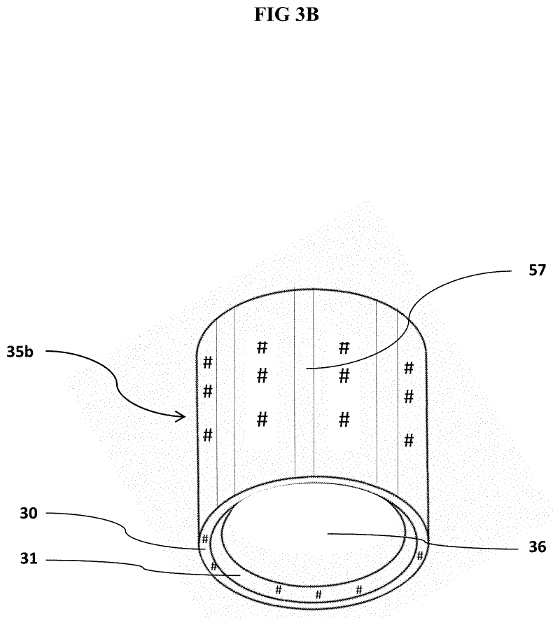

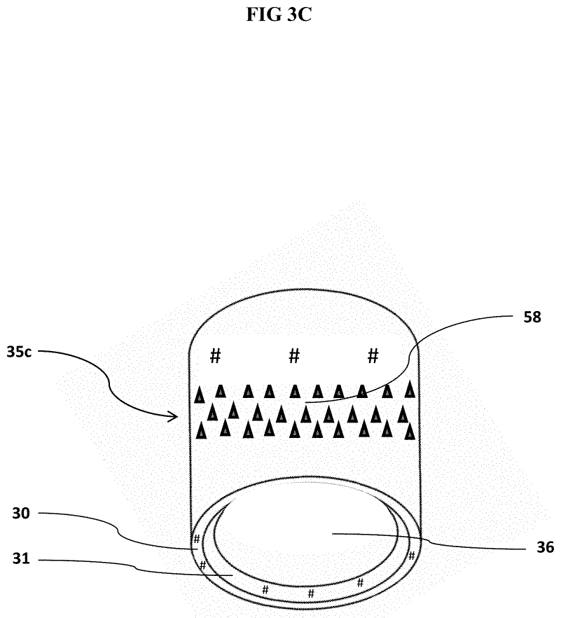

FIGS. 3, 3A, 3B, and 3C explain different varieties of the meshwork-contacting element surface. In one embodiment, FIGS. 3A-3B have a sort of porous or having perforated openings on the meshwork-contacting element. A balloon or surface-medicated balloon is inserted through the lumen 36 of the applicator and positioned at inner side of the meshwork-contacting element so that certain bioactive agent on the surface-medicated balloon may contact the meshwork through screen-type surface, altering-type surface, porous surface or the same of the meshwork-contacting element 35. FIG. 3 shows one embodiment of the meshwork-contacting element having a solid metallic surface 55. FIG. 3A shows one embodiment of the meshwork-contacting element having a screen-type surface 56. FIG. 3B shows one embodiment of the meshwork-contacting element having an altering-type surface 57. FIG. 3C has somewhat of a coarse surface 58 that when the meshwork-contacting element is rotated, it allows the meshwork to be ablated or cut away to create an opening.

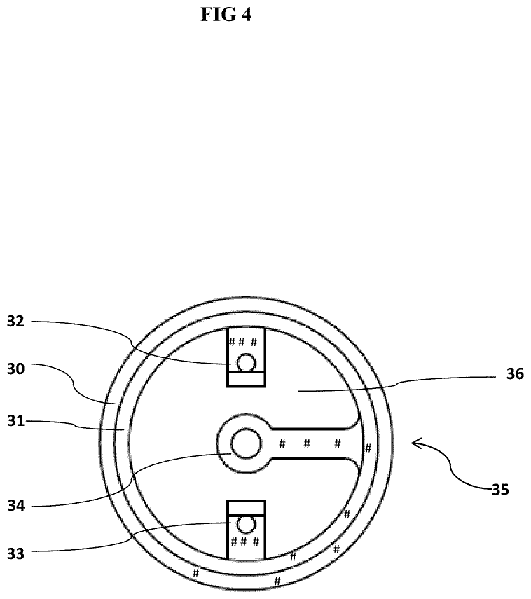

FIGS. 3 to 4 show an embodiment of different views of the meshwork-contacting element 35. In one embodiment, at least a portion of exterior 30 of the element 35 is made of a non-metallic material, a porous material, a perforated material, or a material with substantial openings. In another embodiment, part of the interior of the element is made of a metallic inner ring 31, constructed in accordance with one embodiment as described below. In one particular embodiment, the outer ring and the inner ring is an integral ring having an outer surface and an inner surface.

Referring in particular to FIGS. 3, 4, and 5, in one embodiment, the element 35 is generally in the form of a hollow cylinder, with positively charged connector 32 and a negatively charged connector 33 within the lumen 36 or shaft of the applicator, whereas the connectors are connected to a remote radiofrequency generator 46 or other energy sources. As mentioned in one embodiment, within the shaft of the applicator is a connecting means 34 by allowing the connectors from connectors 32, 33 to the RF generator or other energy sources through the lumen of the element 35.

As best seen in FIG. 4, the element 35 is generally circular, round, or pseudo-round in shape. In modified embodiments, it may be shaped in other suitable manners, such as an oval or the like, as needed to ablate an opening throughput hole adapting for effective aqueous entrance and transmission through altered trabecular meshwork. In a further embodiment, the exterior surface of the element may be rough or spiked to rupture the adjacent trabecular meshwork (as shown in FIG. 3C). The element 35 may comprise of other appropriate shape, size, or configurations.

Preferably, the entire exposed surface of the element 35 is biocompatible and tissue compatible so that the interaction/irritation between its exterior surface and the surrounding tissue or aqueous is minimized. In modified embodiments, selected portions or surfaces of the element 35 may comprise a biocompatible and/or tissue compatible material, as needed or desired.

The element 35 of the illustrated embodiment may be demonstrated in a wide variety of manners. In an exemplary embodiment, the element 35 has a length between about 0.5 millimeters (mm) to about over 1 centimeter (cm), depending on where the device is inserted within the meshwork. The outside diameter of the element 35 may range from 10 micrometers (μm) to about 600 micrometers (μm) or more. In other embodiments, the element 35 may be demonstrated in modified manners with efficacy, as required or desired, giving due consideration to the goals of achieving one or more of the benefits and advantages as taught or suggested herein.

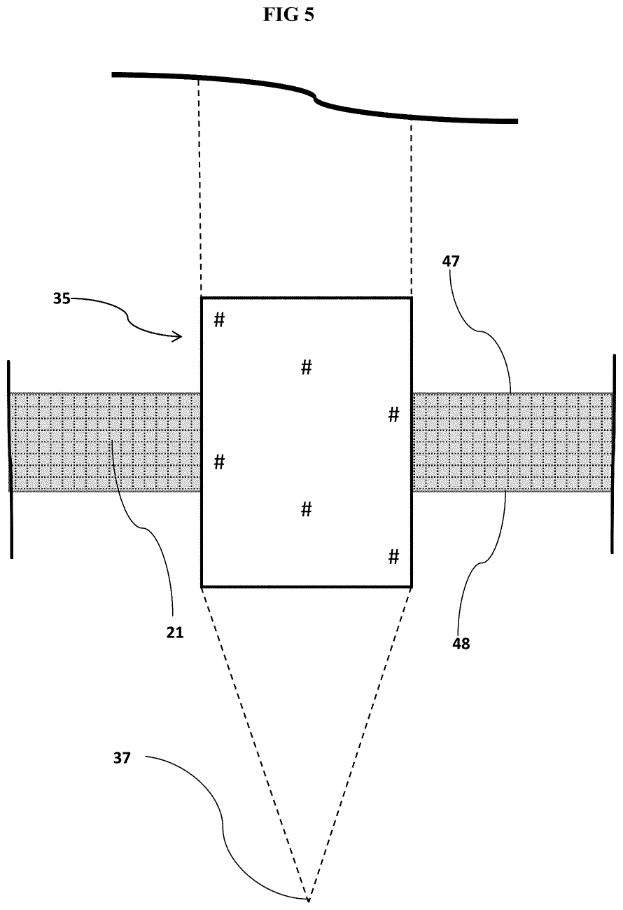

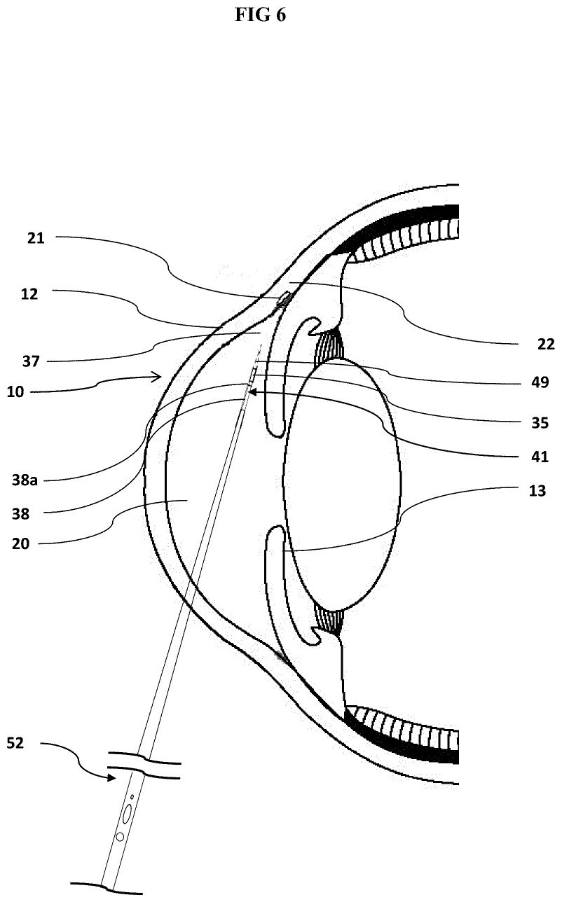

As best seen in FIG. 6 in use, the applicator piercing member 49 along with the element 35 of the applicator 40 is inserted into the anterior chamber 20 of the eye 10 at the trabecular meshwork 21. As illustrated in FIG. 5 after the meshwork-contacting element 35 is in position as desired, the trabecular meshwork interior side or surface 47 faces the anterior chamber 20 and the trabecular meshwork exterior side or surface 48 faces Schlemm's canal 22.

From FIG. 6, it is seen that the piercing element on a handpiece or applicator is inserted by a practitioner in clinical use. In an exemplary embodiment of the trabecular meshwork surgery, the patient is placed in the supine position, prepped, draped and administered anesthesia. In one embodiment, a small (less than 1 mm in a “Sub one” surgery) self-sealing incision is made in the cornea 12. The piercing member of the applicator 40 is advanced through the corneal incision across the anterior chamber 20 held in the irrigated apparatus under gonioscopic lens or endoscopic guidance. As illustrated in FIG. 6, the meshwork-contacting element 35 is connected to the stainless steel tube 38 of the applicator 40 via a tapered cutting-edge (Bolster) 38a.

The piercing member 49 of the applicator 40 is used to make an incision in the trabecular meshwork 21 and create an opening using the element to advantageously allow the fluid to flow through in a one-step procedure. This one-step procedure may include turning on the RF power for 1-2 seconds or more. In some embodiments, the element is rotated briefly or momentarily to assist creating a throughput hole on the altered trabecular meshwork. The applicator 40 is then withdrawn from the eye and the surgery is concluded, without leaving an implant or stent in place.

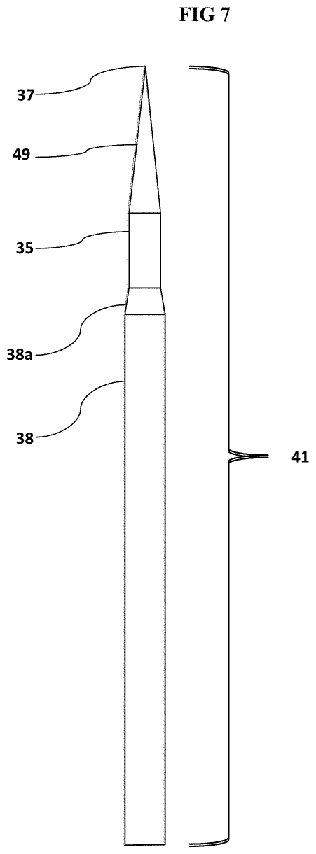

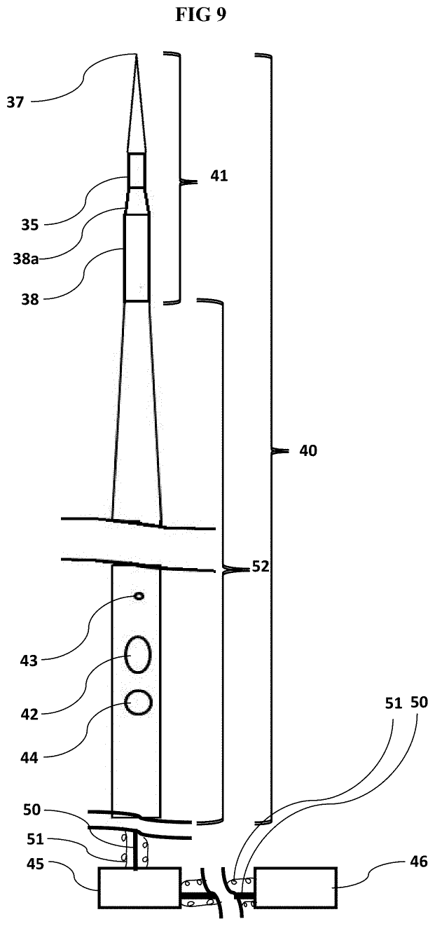

FIGS. 7-9 show the applicator and setup for surgical operations. FIG. 7 shows the tip section 41 of the applicator 40. The applicator also includes the piercing member 49, the element 35, and the applicator body 52. FIG. 8 is a cross-sectional view of the applicator 40, whereas it shows a rotatable member 50 and a tubular shaft 51 within the applicator body 52. FIG. 9 incorporates an external electric conductor 45 that is connected to an RF generator 46 for supplying energy to the element 35 when needed. FIG. 9 also shows the tip section 41 and the entire applicator 40 which includes but is not limited to the power on-off button 42 and optionally light indicator 43 which displays the power. The rotational button 44 may conveniently be located on the applicator 40.

In FIG. 6, element 35 of the applicator 40 may optionally be rotated from 1-90 degrees or more (either or both ways) while being inserted in the meshwork. When the power is turned on or turned off, the element may also be rotated 1-90 degrees or more. This method may use 1-3 or more cycles. The opening can be expanded over 150-200% of the original opening using rotational mechanism as well as heating/cryogenic mechanism.

The external RF current generator means has the capability to supply RF current by controlling the time, power, and temperature through an optional separate closed-loop temperature control means (not shown here), which is well known to those who are familiar with the technology. The patient is connected to the RF generator means through a DIP electrode to form a closed-loop RF current system. Therefore, RF energy is applied and delivered to the targeted tissue region, through the meshwork-contacting element of this invention. The radiofrequency energy current in this invention is preferably within the range of 50 to 2,000 kHz. In one embodiment, by simultaneously applying RF energy to the electrode element (i.e., the meshwork-contacting element 35) and by applying the rotational therapy, the trabecular meshwork tissue can be altered or ablated as desired or described.

In a particular embodiment, the material for the RF current delivery medium of this invention consists of conductive metals such as platinum, iridium, gold, silver, stainless steel, Nitinol, or an alloy of the conductive metals.

For the piercing member 49, the sharp tip 37 may be a surgical needle or a sharp embodiment as needed. In some embodiments, at least one pharmaceutical ingredient or bioactive agent may be added to the meshwork-contacting element of the applicator 40 in order for the at least one pharmaceutical ingredient or bioactive agent to contact and stick to the meshwork. The at least one pharmaceutical ingredient or bioactive agent may be sprayed or painted on and mixed with an adhesive or sticky substance in order for it to stay on the meshwork longer. In one embodiment, the outside diameter (OD) of the meshwork-contacting element 35 can be sized 0.1-0.3 mm or bigger.

In some embodiment, the meshwork-contacting element 35 may provide cryogenic energy, instead of heat energy as described above, to ablate the trabecular meshwork for enhancing the aqueous outflow once a hole or spacious throughput was created via the cryogenic energy. The cryogenic energy can be supplied via, for example, liquid nitrogen or any cryogenic fluid, through the meshwork-contacting element 35 as taught elsewhere. U.S. Pat. No. 10,251,693 to Newell et al, disclosed an ablation assembly including a controller assembly and a cryogenic ablation catheter, entire content of which are included herein by reference.

In one embodiment, the invention relates to a method of surgically altering trabecular meshwork of an eye to create a throughput hole on the meshwork comprising: (a) inserting a meshwork-contacting element of an applicator into the meshwork; (b) contacting the meshwork, (c) the meshwork-contacting element provides energy or mechanical force to the contacted trabecular meshwork, and (d) thereafter removing the applicator from the eye.

In some embodiment, the surface of the meshwork-contacting element 35 may be coated with a pharmaceutical ingredient (or therapeutic drug) that can be released once contacting the desired trabecular meshwork site. In another embodiment, the drug can be releasably stored within the meshwork-contacting element 35. Once at the desired trabecular meshwork site, the therapeutic drug is released. In this particular embodiment, the meshwork-contacting element 35 may be porous or equipped with a plurality of small holes or openings (regular or irregular shapes/configurations) for releasing the drug. Drug of the present invention may include, but not limited to, immunosuppressive drugs which can be classified into five groups: glucocorticoids, cytostatics, antibodies, drugs acting on immunophilins, and other drugs. In one embodiment, the meshwork-contacting element 35 can be a balloon or drug-coated balloon such as cited in the cardiovascular balloon catheter field. A balloon catheter is a type of “soft” catheter with an inflatable “balloon” at its tip which is used during a catheterization procedure to enlarge a narrow opening or passage within the body. U.S. Pat. No. 10,314,948 by Michal et al. disclosed “local delivery of water-soluble or water-insoluble therapeutic agents to the surface of body lumen”, entire contents of which are included herein for reference.

In one embodiment, the invention relates to a method of surgically delivering a pharmaceutical ingredient to trabecular meshwork of an eye comprising: (a) inserting a meshwork-contacting element of an applicator into the meshwork; wherein the surface of the meshwork-contacting element is coated with a pharmaceutical ingredient (or therapeutic drug) that can be released once contacting the target trabecular meshwork site; (b) contacting the meshwork; and thereafter removing the applicator from the eye. In another embodiment, the drug can be releasably stored within the meshwork-contacting element. Once at the target trabecular meshwork site, the therapeutic drug is released. In this particular embodiment, the meshwork-contacting element may be porous or equipped with a plurality of small holes or openings for releasing the drug,

From the foregoing description, it should now be appreciated that an ablation apparatus system for the tubular organs, atherosclerosis, and the treatment of vascular tissues, comprising a suitable energy source and/or a rotational pressure therapy has been disclosed. While the invention has been described with reference to a specific embodiment, the description is illustrative of the invention and is not to be construed as limiting the invention. Various modifications and applications may occur to those who are skilled in the art, without departing from the true spirit and scope of the invention, as described by the appended claims.
Eye 10
Sclera 11
Cornea 12
Iris 13
Pupil 14
Limbus 15
Ciliary body 16
Choroid 17
Retina 18
Optic nerve 19
Anterior chamber 20
Trabecular meshwork 21
Schlemm's canal 22
Aqueous veins 23
Conjunctive 24
Chamber angle 25
Lens 26
Outer ring of the element 30
Inner ring of the element 31
Positive-charged connector 32
Negative-charged connector 33
Metallic guide (centering) 34
Meshwork-contacting element 35
Lumen 36
Sharp tip 37
Cutting-Edge(Bolster) 38a
Stainless steel tube (Heel of Tip) 38
Applicator 40
Tip section of applicator 41
Power button (continue to push for device to stay on) 42
Light indicator 43
Rotational button 44
Electric conductor 45
RF generator 46
Interior surface of meshwork 47
Exterior surface of meshwork 48
Piercing member of the applicator 49
Rotatable member 50
Tubular shaft 51
Applicator body 52
Solid surface 55
Screen-Type surface 56
Altering-Type surface 57
Coarse surface 58

What is claimed is:

1. A method of surgically altering trabecular meshwork of an eye to create at least one throughput hole on said meshwork comprising: inserting a meshwork-contacting element of an applicator into said meshwork and contacting said meshwork, wherein said meshwork-contacting element provides energy or mechanical force to the contacted trabecular meshwork, and thereafter removing the applicator from the eye.

2. The method of claim 1, wherein said meshwork-contacting element is located at a tip section of said applicator.

3. The method of claim 1, wherein said energy is heat energy or cryogenic energy.

4. The method of claim 1, wherein said energy is an alternating heat energy and cryogenic energy cycle.

5. The method of claim 1, wherein the applicator further comprises a piercing member.

6. The method of claim 4, wherein the piercing member is a self-trephining member.

7. The method of claim 1, wherein an exterior surface of the meshwork-contacting element is made of metallic material or energy generating material.

8. The method of claim 1, wherein an exterior surface of the meshwork-contacting element is coated with a pharmaceutical active ingredient.

9. The method of claim 1, wherein an exterior surface of the meshwork-contacting element is porous or having perforated openings.

10. The method of claim 1, wherein an exterior surface of the meshwork-contacting element is a coarse surface or having sharp points so that when the meshwork-contacting element is rotated, the meshwork is ablated or cut away to create the throughput hole.

11. The method of claim 1, wherein a procedure for inserting the meshwork-contacting element into said meshwork is via an ab interno procedure.

12. A method of surgically delivering a pharmaceutical ingredient to trabecular meshwork of an eye comprising: inserting a meshwork-contacting element of a hollow applicator into the meshwork and contacting the meshwork, wherein a surface-medicated balloon is inserted through a lumen of the applicator to the meshwork-contacting element, wherein the surface-coated pharmaceutical ingredient or therapeutic drug is released by inflating the balloon outwardly toward the contacted meshwork, and thereafter removing the applicator from the eye.

13. The method of claim 12, wherein the meshwork-contacting element may be equipped with a plurality of small holes or openings for allowing the balloon surface to contact the target meshwork.

14. The method of claim 12, wherein a procedure for inserting the meshwork-contacting element into said meshwork is via an ab interno procedure.
